(19) United States
(12) Patent Application Publication
(10) Pub. No.: US 2012/0150027 A1
(43) Pub. Date: Jun. 14, 2012

(54) METHODS AND SYSTEMS FOR ENDOBRONCHIAL DIAGNOSIS AND TREATMENT

(75) Inventors: Surag Mantri, Sunnyvale, CA (US); Srikanth Radhakrishnan, Cupertino, CA (US)

(73) Assignee: PULMONX CORPORATION, Redwood City, CA (US)

(21) Appl. No.: 13/174,649

(22) Filed: Jun. 30, 2011

Related U.S. Application Data
(60) Provisional application No. 61/360,814, filed on Jul. 1, 2010.

(51) Int. Cl. A61B 5/08; A61B 6/03; A61M 16/00; A61B 6/00

(52) U.S. Cl. 600/425; 600/529; 600/431; 128/207.14

(57) ABSTRACT

A method of assessing a lung compartment of a patient may involve: advancing a diagnostic catheter into a lung airway leading to a first sub-compartment of the lung compartment; inflating an occluding member disposed on the diagnostic catheter to form a seal with a wall of the airway and thus isolate the first sub-compartment; introducing a diagnostic gas into the first sub-compartment; and recording a perfusion value of the diagnostic gas within the first sub-compartment.

### CROSS-REFERENCE TO RELATED APPLICATION

This application claims priority under 35 U.S.C. §119(e) to U.S. Provisional Patent Application Ser. No. 61/360,814, entitled Methods and Systems for Endobronchial Diagnosis and Treatment, filed Jul. 1, 2010, the full disclosure of which is hereby incorporated by reference.

### BACKGROUND OF THE INVENTION

1. Field of the Invention

This invention relates generally to methods for diagnosis and treatment of lung disease.

2. Description of the Related Art

Chronic obstructive pulmonary disease (COPD), including emphysema and chronic bronchitis, is a significant medical problem currently affecting around 16 million people in the U.S. alone (about 6% of the U.S. population). In general, two types of diagnostic tests are performed on a patient to determine the extent and severity of COPD: 1) imaging tests; and 2) functional tests. Imaging tests, such as chest x-rays, computerized tomography (CT) scans, Magnetic Resonance Imaging (MRI) images, perfusion scans, and bronchograms, provide a good indicator of the location, homogeneity and progression of the diseased tissue. However, imaging tests do not provide a direct indication of how the disease is affecting the patient's overall lung function and respiration. Lung function can be better assessed using functional testing, such as spirometry, plethysmography, oxygen saturation, and oxygen consumption stress testing, among others. Together, these imaging and functional diagnostic tests are used to determine the course of treatment for the patient.

One of the emerging treatments for COPD involves the endoscopic introduction of endobronchial occluders or endobronchial one-way valve devices (“endobronchial valves” or “EBVs”) into pulmonary airways to cause atelectasis (i.e., collapse) of a diseased/hyperinflated lung compartment, thus reducing the volume of that lung portion and allowing healthier lung compartments more room to breathe and perhaps reducing pressure on the heart. Examples of such a method and implant are described, for example, in U.S. patent application Ser. No. 11/682,986 and U.S. Pat. No. 7,798,147, the full disclosures of which are hereby incorporated by reference. One-way valves implanted in airways leading to a lung compartment restrict air flow in the inhalation direction and allow air to flow out of the lung compartment upon exhalation, thus causing the adjoining lung compartment to collapse over time. Occluders block both inhalation and exhalation, also causing lung collapse over time.

It has been suggested that the use of endobronchial implants for lung volume reduction might be most effective when applied to lung compartments which are not affected by collateral ventilation. Collateral ventilation occurs when air passes from one lung compartment to another through a collateral channel rather than the primary airway channels. If collateral airflow channels are present in a lung compartment, implanting a one-way valve or occluder might not be as effective, because the compartment might continue to fill with air from the collateral source and thus fail to collapse as intended. In many cases, COPD manifests itself in the formation of a large number of collateral channels caused by rupture of alveoli due to hyperinflation, or by destruction and weakening of alveolar tissue.

An endobronchial catheter-based diagnostic system typically used for collateral ventilation measurement is disclosed in U.S. Patent Publication No. 2003/0051733 (hereby incorporated by reference), wherein the catheter uses an occlusion member to isolate a lung segment and the instrumentation is used to gather data such as changes in pressure and volume of inhaled/exhaled air. Current state of the art methods for collateral ventilation measurement are disclosed in U.S. Pat. No. 7,883,471 and U.S. Patent Publication Nos. 2008/0027343 and 2007/0142742 (all of which are hereby incorporated by reference), in which an isolation catheter is used to isolate a target lung compartment and pressure changes therein are sensed to detect the extent of collateral ventilation. The applications also disclose measurement of gas concentrations to determine the efficiency of gas exchange within the lung compartment. Similar methods are disclosed in PCT Application No. WO2009135070A1 (hereby incorporated by reference), wherein gas concentration changes in a catheter-isolated lung portion allow collateral ventilation to be determined.

In addition to the detection of collateral channels, it would be desirable to be able to measure other disease-related parameters. For example, the ELVR method disclosed above is typically used to seal off an entire lobe, segment, or multiple lobes or segments of a lung. In some cases, however, lung disease might affect parts of a lobe or segment of a lung differently than other parts of the same lobe or segment. In some cases where an entire lobe is collapsed using EBVs, for example, some amount of healthy lung tissue (for example, one or more segments of the lobe) is subject to the same amount of lung collapse as the unhealthy tissue. Further, the health and suitability of lung tissue may not be determined by presence of collateral ventilation alone. Other factors, such as perfusion rate, are useful in determining the functionality of lung tissue. Thus, a need exists to assess and compare lung segments and in some cases sub-segments with parameters in addition to collateral ventilation. At least some of the methods disclosed below will address this need.

Furthermore, once a patient is treated with a pulmonary intervention of some kind, it would be useful for the patient to have improved methods and devices for rehabilitating one or both lungs. It would be cost-effective for a patient to be trained using the same machine that performed the diagnosis. Indeed, it would be cost-effective for the hospital to be able to use a diagnostic machine as a training tool for the respiratory therapy needs of not only lung-reduction patients, but also patients in all health stages. The training component of the tool would use similar principles and methods as the diagnostic component to maximize efficiency, but would be easy for both the patient and the administrator to use. At least some of the methods disclosed below will address this need.

### SUMMARY OF THE INVENTION

In one aspect, a method of assessing a lung compartment of a patient may involve: advancing a diagnostic catheter into a lung airway leading to a first sub-compartment of the lung compartment; inflating an occluding member disposed on the diagnostic catheter to form a seal with a wall of the airway and thus isolate the first sub-compartment; introducing a diagnostic gas into the first sub-compartment; and recording a perfusion value of the diagnostic gas within the first sub-compartment. As mentioned previously, the “lung compartment” may in some cases be a lung lobe, with the “sub-compartment” being a segment within the lobe. In other embodiments, the “lung compartment” may be a lung segment, and the “sub-compartment” may be a sub-segment within the segment. In general, the methods herein may be used either for assessing a lung lobe, in which case one or more segments of that lobe are assessed, or for assessing a lung segment, in which case one or more sub-segments of that segment are assessed. The method may also be used to assess an entire lung, in which case the lung is the “compartment” and the lobes of the lung are the “sub-compartments.” Alternatively, the method may also be used to assess on a smaller scale, with a sub-segment being the “compartment” and a smaller portion of the sub-segment being the “sub-compartment.”

Optionally, in some embodiments, the method may further involve: deflating the occluding member; repositioning the diagnostic catheter in an airway leading to a second sub-compartment of the lung compartment; inflating the occluding member to isolate the second sub-compartment; introducing the diagnostic gas into the second sub-compartment; and recording a perfusion value of the diagnostic gas within the second sub-compartment. In some embodiments, the method may further involve comparing the perfusion values of the first and second sub-compartments. Optionally, the method may also involve assessing homogeneity of disease within the lung compartment based on the compared perfusion values.

In some embodiments, the diagnostic gas may comprise a radioactive isotope. In such embodiments, the recording step may involve using an imaging system to determine the rate of perfusion of the diagnostic gas within the first sub-compartment. In one embodiment, for example, the imaging system comprises a computed tomography (CT) scanner, and the recording step involves capturing one or more CT images.

In another aspect, a method of assessing homogeneity of disease within a lung compartment of a patient may involve: advancing a diagnostic catheter into a lung airway leading to a first sub-compartment of the lung compartment; inflating an occluding member disposed on the diagnostic catheter to form a seal with a wall of the airway and thus isolate the first sub-compartment; introducing a diagnostic gas into the first sub-compartment; recording a first perfusion value of the diagnostic gas within the first sub-compartment; deflating the occluding member; repositioning the diagnostic catheter in an airway leading to a second sub-compartment; inflating the occluding member to isolate the second sub-compartment; introducing the diagnostic gas into the second sub-compartment; recording a second perfusion value of the diagnostic gas within the second sub-compartment; and assessing homogeneity of disease within the lung compartment by comparing the first and second perfusion values. In one embodiment, the lung compartment may comprise a lobe of a lung, and the sub-compartments may comprise segments within the lobe. In an alternative embodiment, the lung compartment may comprise a segment of a lung, and the sub-compartments may comprise sub-segments within the segment.

In another aspect, a method for helping a patient rehabilitate a lung after a pulmonary procedure has been performed on the lung may involve: introducing a catheter into the patient's mouth, where the catheter comprises a breathing tube having a distal end configured to be held within the mouth and a proximal end configured to be attached to a console; displaying a first waveform on the console; displaying a second waveform on the console; and instructing the patient to alter a breathing pattern based on a comparison of the first and second waveforms.

In some embodiments, the first waveform may represent a desired breathing pattern, and the second waveform may represent a measured breathing pattern of the patient. Optionally, some embodiments may further include displaying a correlation value between first and second waveforms on the console. In one embodiment, the first waveform may be derived from values obtained in the general population.

Further aspects and embodiments of the present invention are set forth in greater detail below, in reference to the attached drawing figures.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 shows a diagnostic or assessment catheter used in the disclosed methods of the present invention.

FIG. 2 shows the placement of the catheter shown in FIG. 1 in the lung.

FIG. 3 shows a console configured to receive the catheter shown in FIG. 1.

FIG. 4 is a flow diagram illustrating one embodiment of the present invention.

FIG. 5 is a waveform display according to one embodiment of the present invention.

### DETAILED DESCRIPTION

Although the detailed description contains many specifics, these should not be construed as limiting the scope of the invention but merely as illustrating different examples and aspects of the invention. Various modifications, changes and variations may be made in the disclosed embodiments without departing from the spirit and scope of the invention.

The present application provides methods and systems for targeting, accessing and diagnosing diseased lung compartments. Such compartments could be an entire lobe, a segment, a sub-segment or any such portion of the lung. Diagnosis is achieved in the disclosed embodiments by isolating a lung compartment to obtain various measurements to determine lung functionality. Though COPD is mentioned as an example, the applicability of these methods for treatment and diagnosis is not limited to COPD, but can be applicable to any lung disease.

The methods are minimally invasive in the sense that the required instruments are introduced through the mouth (or a tracheostomy or other external opening in connection with an airway), and the patient is allowed to breathe normally during the procedures. The methods involve detecting the presence or characteristics (e.g., concentration or pressure) of one or more naturally occurring or introduced gases to determine the presence of collateral ventilation, or may involve measurement of oxygen saturation of tissue.

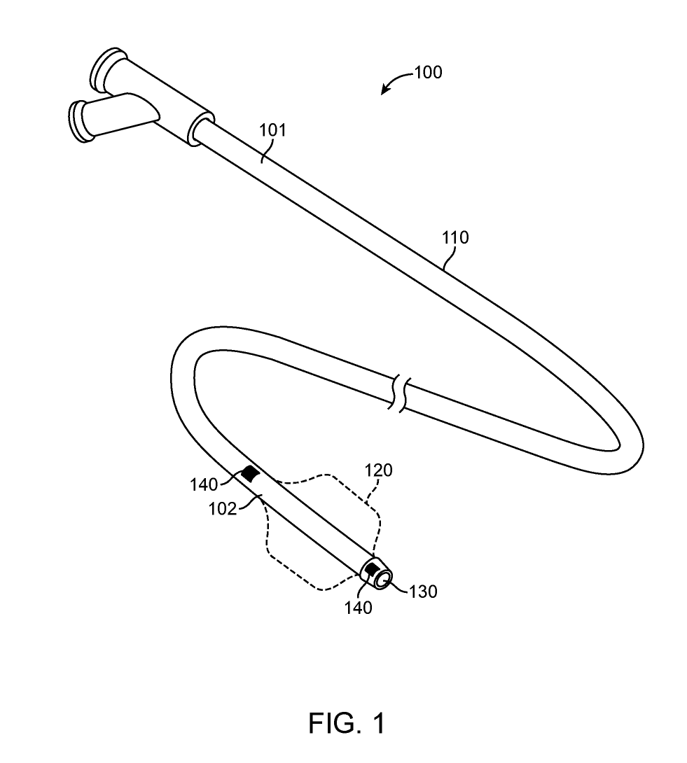

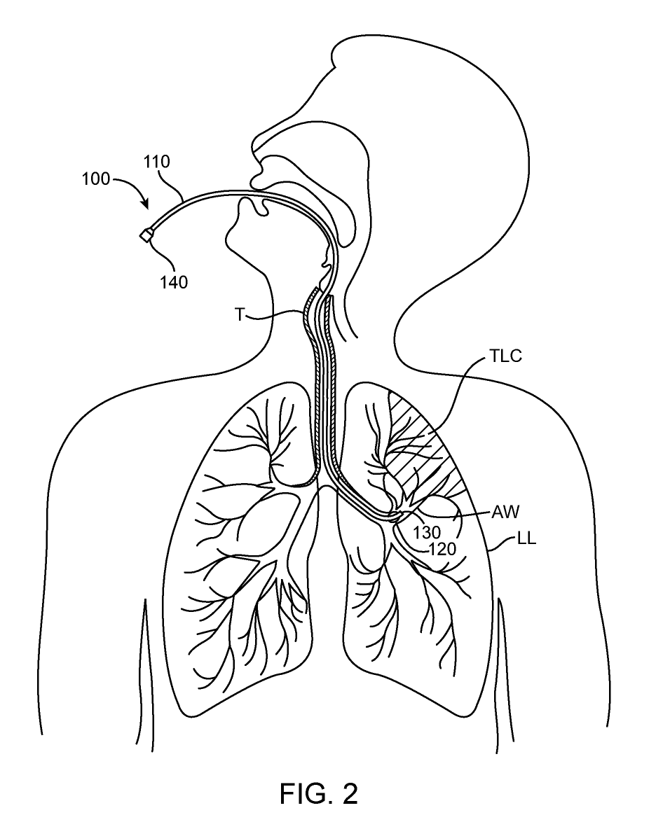

In some embodiments, isolation of the lung comprises sealingly engaging a distal end of a catheter in an airway feeding a lung compartment, as shown in FIGS. 1 and 2. Such a catheter has been disclosed in co-pending published U.S. patent application Ser. No. 10/241,733, which is incorporated herein by reference. As shown in FIG. 1, the catheter 100 comprises a catheter body 110, and an expandable occluding member 120 on the catheter body. The catheter body 110 has a distal end 102, a proximal end 101, and at least one lumen 130, extending from a location at or near the distal end to a location at or near the proximal end.

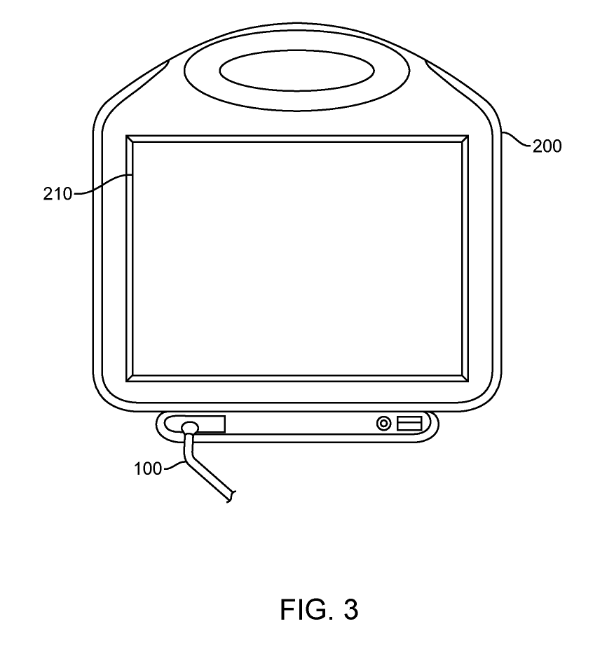

The proximal end of catheter 100 is configured to be coupled with an external control unit (or “console,” as in FIG. 3), and optionally comprises an inflation port (not shown). The distal end of catheter 100 is adapted to be advanced through a body passageway such as a lung airway. The expandable occluding member 120 is disposed near the distal end of the catheter body and is adapted to be expanded in the airway which feeds the target lung compartment. In one embodiment, the occluding member 120 is a compliant balloon made of transparent material. The transparent material allows visualization using the bronchoscope through the balloon. The occluding member 120 is inflatable via a syringe that is configured to be coupled to the inflation port. Optionally, catheter 100 comprises visual markers at the proximal and distal ends of the balloon to identify the location of the occluding member 120 within the airway prior to inflation. The occluding member 120 material inflates and seals with inflation pressures between 5-20 psi to prevent balloon migration within the airway. This inflation pressure also aids the occluding member 120 in maintaining a symmetrical configuration within the airway, thereby ensuring that the catheter (which is centered within the occluding member 120) will remain centered within the airway. The occluding member 120 material and attachment are also configured to minimize longitudinal movement of the occluding member 120 relative to the catheter body 110 itself. To accommodate the higher inflation pressure, the occluding member 120 is made of a polyurethane such as Pellethane 80A, but can be made of any material that is configured to maintain structural integrity at a high inflation pressure.

Additionally and optionally, catheter 100 may further include at least one gas sensor 140 located within or in-line with the lumen 130 for sensing characteristics of various gases in air communicated to and from the lung compartment. The sensors may comprise any suitable sensors or any combination of suitable sensors, and are configured to communicate with control unit 200. Examples of sensors include pressure sensors, temperature sensors, air flow sensors, gas-specific sensors, or other types of sensors. As shown in FIG. 1, the sensors 140 may be located near the distal end 102 of the catheter 100. Alternatively, the sensors 140 may be located at any one or more points along the catheter 100, or in-line with the catheter 100 and within the control unit with one or more measuring components.

As shown in FIG. 2, at least a distal portion of the catheter body 110 is adapted to be advanced into and through the trachea (T). The catheter may optionally be introduced through or over an introducing device such as a bronchoscope. The distal end 102 of the catheter body 110 can then be directed to a lung lobe (LL) to reach an airway (AW) which feeds a target lung compartment (TLC), which is to be assessed. When the occluding member 120 is expanded in the airway, the corresponding compartment is isolated with access to and from the compartment provided through the lumen 130.

Referring now to FIG. 3, the proximal end of the catheter 100 is configured to be coupled with a control unit (or “console”) 200. The control unit 200 comprises one or more measuring components (not shown) to measure lung functionality. The measuring components may take many forms and may perform a variety of functions. For example, the components may include a pulmonary mechanics unit, a physiological testing unit, a gas dilution unit, an imaging unit, a mapping unit, a treatment unit, a pulse oximetry unit or any other suitable unit. The components may be disposed within the control unit 200, or may be attached to the unit 200 from an external source. The control unit 200 comprises an interface for receiving input from a user and a display screen 210. The display-screen 210 will optionally be a touch-sensitive screen, and may display preset values. Optionally, the user will input information into the control unit 200 via a touch-sensitive screen mechanism. Additionally and optionally, the control unit 200 may be associated with external display devices such as printers or chart recorders. At least some of the above system embodiments will be utilized in the methods described below.

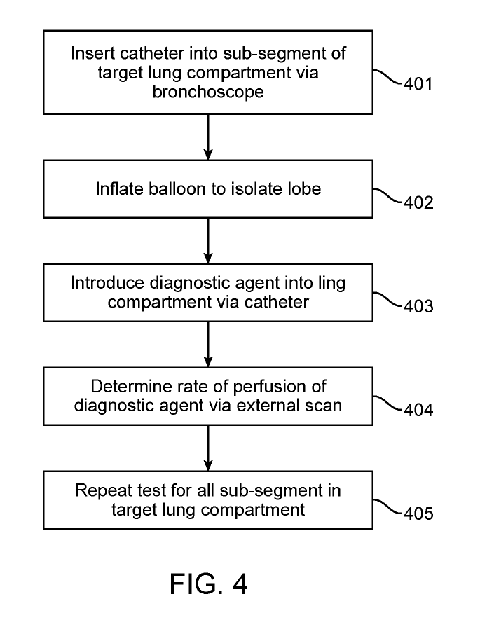

Referring now to FIG. 4, in one embodiment, the system of FIGS. 1 and 3 may be used in a method 400 to assess one or more sub-compartments of a lung compartment. As mentioned previously, the lung compartments and sub-compartments may be any portions of the lung. One assessment that may be made is to compare two or more sub-compartments regarding how diseased they are relative to one another. Such an assessment may be made, for example, in deciding whether to treat a given compartment or sub-compartment with an EBV (or multiple EBVs) or some other implant or other treatment. For example, a pulmonologist may assess multiple segments of a lung lobe and decide that some are relatively healthy while others are much more diseased. The pulmonologist may then decide to use EBVs to collapse the most unhealthy segments and to not treat the healthier segments. In contrast, in another patient a pulmonologist may assess multiple segments of a lobe, may find that all of them are relatively diseased, and thus may decide to collapse the entire lobe using EBVs. One way to assess lung sub-compartments, for example, is by introducing a diagnostic agent into a sub-compartment and thereafter monitoring the sub-compartment to determine perfusion status of the sub-compartment.

Pulmonologists sometimes refer to a lung compartment as having either a “homogeneous” or “heterogeneous” distribution of disease. “Homogeneous” and “heterogeneous” are terms of art, typically used to describe distribution of disease within a lung or lung lobe. In other words, if a lobe of a lung has relatively the same amount of disease throughout, it may be called “homogeneous,” while if it has areas that are significantly more diseased than other areas, it may be called “heterogeneous.” However, these terms are subjective and thus should not be interpreted to limit the scope of the claims of this application.

Referring again to FIG. 4, a method 400 for assessing a compartment of a lung may include several steps. In step 401, the catheter is introduced into an airway leading to a sub-compartment of the lung compartment being assessed, as shown in FIG. 2 above. The sub-compartment is then isolated, as seen in step 402, by inflating the occluding member 120 to form a seal with the airway wall. Once the sub-compartment is isolated, as seen in step 403, a diagnostic agent is introduced into the catheter, either via console 200, or via an external port on the catheter. The diagnostic agent is one that is viewable by an imaging device, for example a gas containing radioisotopes that are viewable by using fluoroscopy. Thereafter, in step 404, the rate of perfusion of the diagnostic agent is determined by studying the presence of the gas in other portions of the lung or within the global system. Thereafter, in step 405, the same procedure is repeated for as many sub-compartments within the lung compartment as desired. The rates of perfusion in the sub-compartments may then be compared, if desired, to assess whether some, all or none of the sub-compartments should be treated. For example, if the rates of perfusion for all sub-compartments within the lung compartment are relatively the same, the physician may decide to collapse the entire lung compartment using one or more EBVs, other implants, or other procedures (such as lung volume reduction surgery). Alternatively, if the rates of perfusion of the sub-compartments are significantly different, the physician may decide to treat some sub-compartments individually and not others.

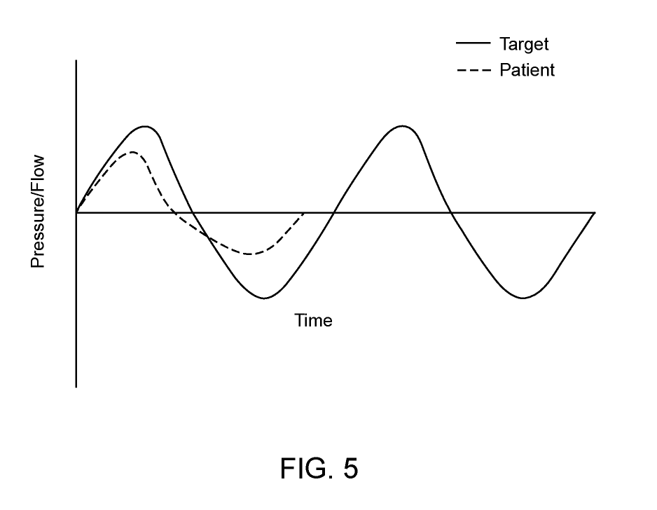

With reference now to FIG. 5, the system described in FIGS. 1-3 (the catheter, the console and/or the data provided by the catheter and console) may also be used to help a patient rehabilitate a lung (or both lungs, or a portion of a lung) after undergoing a pulmonary procedure (EBV placement, lung volume reduction surgery, etc.) Such a function is useful after a lung reduction procedure, but may also be used for other pulmonary rehabilitation purposes, related or unrelated to pulmonary surgery, implant procedures, or the like.

In this method, the console is used to demonstrate a breathing pattern that a patient is encouraged to replicate, and the catheter with console is used to record the actual breathing pattern of the patient. Thus, the patient can compare his/her actual breathing pattern to a desired breathing pattern and can try to alter his/her breathing to bring it closer to the desired pattern. Specifically, in one embodiment, the console 200 is used to produce a general waveform reflecting a breathing pattern. Such a waveform may be obtained, for example, by sampling values from the general population. The waveform, once created, is stored within the memory of the console. When needed, the waveform is displayed on the console 200 as a first waveform. This is shown, for example, in FIG. 5, where the first wave form is the “Target” waveform. The catheter (or any other suitable device in alternative embodiments) is then placed in a patient's mouth, and the patient is encouraged to blow into the catheter. As the patient blows into the catheter, the breathing pattern of the patient is reflected on the console as a second waveform. This is reflected in FIG. 5 as the “Patient” waveform. The patient is encouraged to blow into the catheter in a manner sufficient to have the patient's produced (second) waveform match the first waveform. Optionally, the console 200 displays a correlation value between first and second waveforms to quantify the similarity between the first and second waveforms.

Optionally, the console 200 may be replaced with another device after the first waveform is obtained from the general population. The first waveform may be transposed to another device, such as a video-game console or a hand-held electronic device that has been configured to analyze characteristics of respiration.

Although certain embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any methods need not be performed sequentially.

What is claimed is:

1. A method of assessing a lung compartment of a patient, the method comprising:
advancing a diagnostic catheter into a lung airway leading to a first sub-compartment of the lung compartment;
inflating an occluding member disposed on the diagnostic catheter to form a seal with a wall of the airway and thus isolate the first sub-compartment;
introducing a diagnostic gas into the first sub-compartment; and
recording a perfusion value of the diagnostic gas within the first sub-compartment.

2. The method of claim 1, further comprising:
deflating the occluding member;
repositioning the diagnostic catheter in an airway leading to a second sub-compartment;
inflating the occluding member to isolate the second sub-compartment;
introducing the diagnostic gas into the second sub-compartment; and
recording a perfusion value of the diagnostic gas within the second sub-compartment.

3. The method of claim 2, further comprising repeating the steps for at least a third sub-compartment of the lung compartment.

4. The method of claim 2, further comprising comparing the perfusion values of the first and second sub-compartments.

5. The method of claim 4, further comprising selecting one of the sub-compartments for treatment based on the comparison of perfusion values.

6. The method of claim 1, wherein the diagnostic gas comprises a radioactive isotope.

7. The method of claim 6, wherein the recording step comprises using an imaging system to determine the rate of perfusion of the diagnostic gas within the first sub-compartment.

8. The method of claim 7, wherein using the imaging system comprises recording computed tomography images.

9. The method of claim 1, wherein the lung compartment comprises a lobe of a lung, and wherein the first sub-compartment comprises a segment within the lobe.

10. The method of claim 1, wherein the lung compartment comprises a segment of a lung, and wherein the first sub-compartment comprises a sub-segment within the segment.

11. A method of assessing homogeneity of disease within a lung compartment of a patient, the method comprising:
advancing a diagnostic catheter into a lung airway leading to a first sub-compartment of the lung compartment;
inflating an occluding member disposed on the diagnostic catheter to form a seal with a wall of the airway and thus isolate the first sub-compartment;
introducing a diagnostic gas into the first sub-compartment;
recording a first perfusion value of the diagnostic gas within the first sub-compartment;
deflating the occluding member;
repositioning the diagnostic catheter in an airway leading to a second sub-compartment;
inflating the occluding member to isolate the second sub-compartment;
introducing the diagnostic gas into the second sub-compartment;
recording a second perfusion value of the diagnostic gas within the second sub-compartment; and
assessing homogeneity of disease within the lung compartment by comparing the first and second perfusion values.

12. The method of claim 11, wherein the lung compartment comprises a lobe of a lung, and wherein the sub-compartments comprise segments within the lobe.

13. The method of claim 11, wherein the lung compartment comprises a segment of a lung, and wherein the sub-compartments comprise sub-segments within the segment.

14. The method of claim 11, wherein the diagnostic gas comprises a radioactive isotope.

15. The method of claim 14, wherein the recording steps comprise using an imaging system to determine the rate of perfusion of the diagnostic gas within the sub-compartments.

16. The method of claim 15, wherein using the imaging system comprises recording computed tomography images.

17. A method for helping a patient rehabilitate a lung after a pulmonary procedure has been performed on the lung, the method comprising:
introducing a catheter into the patient's mouth, wherein the catheter comprises a breathing tube having a distal end configured to be held within the mouth and a proximal end configured to be attached to a console;
displaying a first waveform on the console;
displaying a second waveform on the console; and
instructing the patient to alter a breathing pattern based on a comparison of the first and second waveforms.

18. The method of claim 17, wherein the first waveform comprises a desired breathing pattern and the second waveform comprises a measured breathing pattern of the patient.

19. The method of claim 17, further comprising displaying a correlation value between first and second waveforms on the console.

20. The method of claim 17, wherein the first waveform is derived from values obtained in the general population.
